(19) United States
(12) Patent Application Publication
(10) Pub. No.: US 2025/0012883 A1
(43) Pub. Date: Jan. 9, 2025

(54) A METHOD FOR IMAGE PARAMETER COMPARSION IN MAGNETIC RESONANCE IMAGING SIMULATION

(71) Applicant: CORSMED AB, Stockholm (SE)

(72) Inventors: Erik JACOBSSON, Stockholm (SE); Christos XANTHIS, Athens (GR)

(21) Appl. No.: 18/708,770

(22) PCT Filed: Nov. 23, 2022
(86) PCT No.: PCT/SE2022/051093

§ 371 Date: May 9, 2024

(30) Foreign Application Priority Data
Nov. 24, 2021 (SE) 2151426-0

(51) Int. Cl. G01R 33/56; G01R 33/54; G16H 30/40

(52) U.S. Cl. CPC G01R 33/5608; G01R 33/546; G16H 30/40

(57) ABSTRACT

The present invention describes a method in the field of a magnetic resonance imaging (MRI), said method comprising creating an object MR image in an MRI simulator unit: performing an image/parameter comparison of the object MR image and parameter(s) thereof with a second MR image and/or a corresponding set of parameters functioning as a reference: wherein the comparison is based on at least comparing the anatomical position of the object MR image and/or parameter(s) thereof and the second MR image and/or the corresponding set of parameters, said method also comprising providing automatic feedback on the image/parameter comparison and providing the same in a user interface of the MRI simulator unit.

### FIELD OF THE INVENTION

The present invention relates to a method in the field of a magnetic resonance imaging (MRI).

### SUMMARY OF THE INVENTION

The present invention is directed to a method in the field of a magnetic resonance imaging (MRI), said method comprising
creating an object MR image in an MRI simulator unit;
performing an image/parameter comparison of the object MR image and parameter(s) thereof with a second MR image and/or a corresponding set of parameters functioning as a reference;
   wherein the comparison is based on at least comparing the anatomical position of the object MR image and/or parameter(s) thereof and the second MR image and/or the corresponding set of parameters, said method also comprising
providing automatic feedback on the image/parameter comparison and providing the same in a user interface of the MRI simulator unit.

### SPECIFIC EMBODIMENTS OF THE INVENTION

Below some embodiments of the present invention are disclosed and discussed further.

According to one embodiment, the image/parameter comparison is performed between the object MR image and/or parameter(s) thereof and a set of parameters being at least position and/or orientation of the reference plane, preferably both position and orientation of the reference plane. According to yet another specific embodiment, the image/parameter comparison is performed between the object MR image with parameter(s) thereof and a second MR image with a corresponding set of parameters.

As may be noted, a “reference experiment” is used according to the present invention. This “reference experiment” suitably includes the image with a set of “attached” parameters “attached” or only the parameter values as such. Therefore, the comparison according to the present invention may be based on comparing both the images and the parameter sets, e.g. every time at a different percentage (depending on the experiment).

As should be understood from above, the method according to the present invention embodies both an image-to-image comparison or an image parameter-to-image parameter comparison.

In line with the above, according to one embodiment, the comparison is based on at least comparing the anatomical position of the object MR image and the anatomical position of the second MR image. Also this may be based on a direct image comparison or a parameter comparison as further discussed below.

According to another embodiment, the comparison is based on one or more parameters of the object MR image and a corresponding one or more parameters of the second MR image.

As should be understood from above, the method according to the present invention may be based on positions of the two images, or based on certain input parameters or certain output parameters of the two images. Also combinations are possible according to the present invention. To give examples on input alternatives, this may be directed to pulse sequences or other parameters. Examples of pulse sequence parameters, then e.g. the following parameters may be mentioned: time to echo (TE), time to repetition (TR), flip angle, field of view, matrix size, inversion pulse(s), echo train length, diffusion weighting (b values), tissue contrast, spatial resolution. Also other parameters may be used.

To give examples on out parameters, then e.g. the following may be mentioned: parameters relating to the image, kspace, contrast, resolution, noise (SNR), scan time, SAR, etc. It should be noted that some output parameters are obtained before scanning, and others may be obtained after scanning. Examples of output parameters suitable to use and embodiments of the present invention are further mentioned below.

Furthermore, as should be understood, the present invention is directed to comparison of two images. The created image is created in an MRI simulator unit. The second MR image may also be a simulated image or may be a real image.

Furthermore, MRI simulator unit according to the present invention is suitably a computer program unit based on software. The computer unit may e.g. comprise a user interface and software enabling to perform the method according to the present invention.

According to yet another embodiment, the second MR image and/or corresponding set of parameters functioning as a reference is created in the MRI simulator unit as a reference image or wherein the MR simulator unit performs a search to find an already existing MR image or corresponding set of parameters in a platform of the MRI simulator unit as a suitable reference. In this regard it should be noted that the present invention is not limited to use of simulated images, however using at least one such image or a corresponding set of parameters thereof is of most interest.

The comparison according to the present invention may be performed based on different aspects. According to one embodiment of the present invention, the comparison is based on coordinates (X, Y, Z) for at least one position of the object MR image and the second MR image, respectively, optionally for several positions of the object MR image and the second MR image, respectively. In this regard it may be noted that the reference image should contain information with reference to the position of different parts so that the coordinates thereof become a proxy if the object image covers the same or not. Therefore, the coordinates are used as a proxy to compare between images if they cover the same anatomical positions.

According to yet another embodiment of the present invention, the comparison is based on comparing corresponding peripheral portions of the object MR image and the second MR image. One example is by comparison of one or more corners of the images.

The comparison according to the present invention may also be based on additional parameters and not only position. Therefore, according to one aspect of the present invention, the comparison is based on at least one of the following parameters: scan time, image quality/noise/signal to noise ratio, resolution, contrast, artifacts, or SAR, preferably at least contrast. In relation to the above it should be noted that any parameter used in MR image analysis may be used according to the present invention. Other possible parameters to other compare between the images or at least add as an additional part of the analysis are e.g. slice angulation, phase encoding direction; slice gap, slice thickness, phase FOV, frequency FOV, phase coverage, frequency coverage and/or slice anatomy coverage, or any combination thereof.

According to one embodiment of the present invention, the comparison is based on at least scan time, image quality or resolution, or a combination thereof. These parameters or a combination thereof are preferred parameters according to the present invention, when the comparison is based on such parameters.

According to yet another embodiment, the comparison is based on the anatomical position of the object MR image and the anatomical position of the second MR image, and also on at least one parameter of scan time, image quality and resolution, preferably on all of scan time, image quality and resolution.

Moreover, according to yet another embodiment of the present invention, the automatic feedback is provided as a scoring or marking, preferably with a scoring and/or marking for each parameter used in the image comparison.

The core of the present invention is related to providing automatic feedback on an MR image when compared with a reference image. The reference image may be a manually created image, e.g. chosen by a teacher, or may be an image existing in the used MRI simulator and then either chosen by someone or by the platform in itself. In the latter case an ASI engine may be involved in the process for chosen a suitable reference image.

Moreover, and as mentioned, suitably the method according to the present invention is performed in an MR simulator, implying that images are generated in this MR simulator, and it is also here the automatic feedback is generated and provided to the user.

Furthermore, with reference to the image comparison as such, the image position is a key comparison feature used according to the present invention. In addition to image position, as further described above, also many other comparison parameters may be involved according to the present invention. Examples are scan time, image quality/noise/signal to noise ratio, resolution, contrast, artifacts, and SAR. Especially contrast is an additional comparison parameter of interest according to the present invention. This may be used, for example to control different tissue types with different colors. This may then also be used to make certain tissue types and/or tumor tissue very clear with a color distinction or more or less invisible.

1. A method in the field of a magnetic resonance imaging (MRI), said method comprising
creating an object MR image in an MRI simulator unit;
performing an image/parameter comparison of the object MR image and parameter(s) thereof with a second MR image and/or a corresponding set of parameters functioning as a reference;
wherein the comparison is based on at least comparing the anatomical position of the object MR image and/or parameter(s) thereof and the second MR image and/or the corresponding set of parameters,
said method also comprising
providing automatic feedback on the image/parameter comparison and providing the same in a user interface of the MRI simulator unit.

2. The method according to claim 1, wherein the image/parameter comparison is performed between the object MR image and/or parameter(s) thereof and a set of parameters being at least position and/or orientation of the reference plane, preferably both position and orientation of the reference plane.

3. The method according to claim 1, wherein the image/parameter comparison is performed between the object MR image with parameter(s) thereof and a second MR image with a corresponding set of parameters.

4. The method according to claim 1, wherein the comparison is based on at least comparing the anatomical position of the object MR image and the anatomical position of the second MR image.

5. The method according to claim 1, wherein the comparison is based on one or more parameters of the object MR image and a corresponding one or more parameters of the second MR image.
of the object MR image and/or parameter(s) thereof and the second MR image and/or the corresponding set of parameters.

6. The method according to claim 1, wherein the second MR image and/or corresponding set of parameters functioning as a reference is created in the MRI simulator unit as a reference or wherein the MR simulator unit performs a search to find an already existing MR image or corresponding set of parameters in a platform of the MRI simulator unit as a suitable reference.

7. The method according to claim 1, wherein the comparison is based on coordinates (X, Y, Z) for at least one position of the object MR image and the second MR image, respectively, optionally for several positions of the object MR image and the second MR image, respectively.

8. The method according to claim 1, wherein the comparison is based on comparing corresponding peripheral portions of the object MR image and the second MR image.

9. The method according to claim 1, wherein the comparison is based on at least one of the following parameters: scan time, image quality/noise/signal to noise ratio, resolution, contrast, artifacts or SAR, preferably at least contrast.

10. The method according to claim 9, wherein the comparison is based on at least scan time, image quality or resolution, or a combination thereof.

11. The method according to claim 1, wherein the comparison is based on the anatomical position of the object MR image and the anatomical position of the second MR image, and also on at least one parameter of scan time, image quality and resolution, preferably on all of scan time, image quality and resolution.

12. The method according to claim 1, wherein the automatic feedback is provided as a scoring or marking, preferably with a scoring and/or marking for each parameter used in the image comparison.
